(19) United States
(12) Patent Application Publication
(10) Pub. No.: US 2024/0108227 A1
(43) Pub. Date: Apr. 4, 2024

(54) METHODS AND APPARATUSES FOR INTERNAL ORGAN TEMPERATURE MONITORING

(71) Applicant: Honeywell International Inc., Charlotte, NC (US)

(72) Inventors: Manjunath PATTAR, Charlotte, NC (US); Praneesh Kumar YADAV, Charlotte, NC (US); Manjunatha HM, Charlotte, NC (US)

(21) Appl. No.: 18/467,390

(22) Filed: Sep. 14, 2023

(30) Foreign Application Priority Data
Sep. 30, 2022 (IN) 202211056300

(51) Int. Cl. A61B 5/01; A61B 5/00

(52) U.S. Cl. CPC A61B 5/01; A61B 5/0022; A61B 5/0031; A61B 5/7405; A61B 5/742; A61B 5/746

(57) ABSTRACT

Systems and methods for monitoring an internal organ temperature are provided. The internal organ temperature monitoring system includes a sensor system, where the sensor system includes: a tube having a first end and a second end; a temperature sensor in the tube and located at the first end of the tube; a thermal conductive material occupying space between the temperature sensor and the tube; and an electrical connector located at the second end of the tube, coupling the sensor to a controller component. The internal organ temperature monitoring system further includes the controller component configured to retrieve temperature data from the temperature sensor.

### CROSS-REFERENCE TO RELATED APPLICATIONS

This application claims priority pursuant to 35 U.S.C. 119(a) to Indian Application No. 202211056300, filed Sep. 30, 2022, which application is incorporated herein by reference in its entirety.

### FIELD OF THE INVENTION

Exemplary embodiments of the present disclosure relate generally to internal organ temperature monitoring, and more particularly, to methods, apparatuses, and systems for real-time monitoring of an internal organ temperature.

### BACKGROUND

Internal organ temperature is a vital sign of the status of the heath of the body. Measuring and monitoring an internal organ temperature during the diagnosis and treatment of diseases in a hospital/clinic environment are common practice. For example, the infections of wound or diseases would cause the body temperature to fluctuate very quickly. Conventional methods to measure and monitor the temperature usually involve the utilization of a mercury-filled thermometer. More recently, electronic thermometers, such as digital thermometers or infrared thermometers, become more popular in a hospital/clinic environment. Applicant has identified many technical challenges and difficulties associated with monitoring an internal body temperature. Through applied effort, ingenuity, and innovation, many of these identified problems have been solved by developing solutions that are included in embodiments of the present disclosure, many examples of which are described in detail herein.

### BRIEF SUMMARY

Various embodiments described herein relate to components, apparatuses, and systems for monitoring an internal organ temperature.

In accordance with various embodiments of the present disclosure, an internal organ temperature monitoring system is provided. The internal organ temperature monitoring system includes a sensor system, where the sensor system includes: a tube having a first end and a second end; a temperature sensor in the tube and located at the first end of the tube; a thermal conductive material occupying space between the temperature sensor and the tube; and an electrical connector located at the second end of the tube, coupling the sensor to a controller component. The internal organ temperature monitoring system further includes the controller component configured to retrieve temperature data from the temperature sensor.

In some embodiments, the controller component is further configured to transmit data load packages to a remote computing server, RTHMS, hospital workflows, or mobile devices, the data load packages including the retrieved temperature data.

In some embodiments, the controller component further includes visual and acoustic indicators that show respective alerts or alarms with respect to the retrieved temperature data.

In some embodiments, the controller component is further configured to store the retrieved temperature data and show the respective alerts or alarms in an instance in which the retrieved temperature data falls out of a normal temperature range.

In some embodiments, the temperature sensor is an active type sensor with an application specific integrated circuits (ASIC) die, and the active type sensor is powered by the controller component.

In some embodiments, the temperature sensor is a passive type sensor, and the passive type sensor is a thermocouple or a thermistor.

In some embodiments, the tube takes a form of a catheter, and the catheter is made of a flexible gel-based material.

In some embodiments, the sensor system further comprises an insulating seal to insulate the electrical connector and the tube from an ambient environment outside an internal organ, where the insulating seal is located at the second end of the tube.

In some embodiments, the sensor system is configured to be inserted into an internal organ to measure a temperature of the internal organ.

In some embodiments, the sensor system is replaceable and configured to be replaced after a threshold number times of usages.

According to another embodiment, a method for monitoring an internal organ temperature by a controller component is provided. The method includes retrieving temperature data reading from a sensor system, where the sensor system comprising a tube having a first end and a second end; a temperature sensor in the tube and located at the first end of the tube; a thermal conductive material occupying space between the temperature sensor and the tube; and an electrical connector located at the second end of the tube, coupling the sensor to the controller component; determining whether the retrieved temperature data is in a normal temperature range; generating alerts or alarms in an instance in which the retrieved temperature is not in the normal temperature range.

In some embodiments, the method for monitoring the internal organ temperature by the controller component further includes transmitting data load packages to a remote computing server, RTHMS, hospital workflows, or mobile devices, where the data load packages include the retrieved temperature data.

In some embodiments, the method for monitoring the internal organ temperature by the controller component further includes receiving initialization conditions, by the controller component, before retrieving the temperature data reading from the sensor system, where the initialization conditions include a retrieving rate and the normal temperature range.

The foregoing illustrative summary, as well as other exemplary objectives and/or advantages of the disclosure, and the manner in which the same are accomplished, are further explained in the following detailed description and its accompanying drawings.

### BRIEF DESCRIPTION OF THE DRAWINGS

The description of the illustrative embodiments may be read in conjunction with the accompanying figures. It will be appreciated that, for simplicity and clarity of illustration, elements illustrated in the figures have not necessarily been drawn to scale, unless described otherwise. For example, the dimensions of some of the elements may be exaggerated relative to other elements, unless described otherwise. Embodiments incorporating teachings of the present disclosure are shown and described with respect to the figures presented herein, in which:

FIG. 1 illustrates an exemplary analytics and monitoring system in accordance with various embodiments of the present disclosure.

FIG. 2 illustrates an exemplary schematic representation of an exemplary mobile device in accordance with various embodiments of the present disclosure.

FIG. 3 illustrates an exemplary schematic representation of an exemplary remote computing server of an example remote computing platform in accordance with various embodiments of the present disclosure.

FIG. 4 illustrates an exemplary schematic representation of an exemplary controller component of an internal organ temperature monitoring device in accordance with various embodiments of the present disclosure.

FIG. 5 illustrates an exemplary schematic representation of an internal organ temperature monitoring system in accordance with various embodiments of the present disclosure.

FIG. 6 illustrates an exemplary replaceable component of an internal organ temperature sensor system in accordance with various embodiments of the present disclosure.

FIG. 7 illustrates an exemplary diagram illustrating an internal organ temperature sensor system inserted into an internal organ, in accordance with various embodiments of the present disclosure.

FIG. 8 illustrates an exemplary flow diagram illustrating an exemplary method for monitoring internal organ temperature in accordance with various embodiments of the present disclosure.

### DETAILED DESCRIPTION OF THE INVENTION

Some embodiments of the present disclosure will now be described more fully hereinafter with reference to the accompanying drawings, in which some, but not all embodiments of the disclosure are shown. Indeed, these disclosures may be embodied in many different forms and should not be construed as limited to the embodiments set forth herein; rather, these embodiments are provided so that this disclosure will satisfy applicable legal requirements Like numbers refer to like elements throughout.

As used herein, terms such as “front,” “rear,” “top,” etc. are used for explanatory purposes in the examples provided below to describe the relative position of certain components or portions of components. Furthermore, as would be evident to one of ordinary skill in the art in light of the present disclosure, the terms “substantially” and “approximately” indicate that the referenced element or associated description is accurate to within applicable engineering tolerances.

As used herein, the term “comprising” means including but not limited to and should be interpreted in the manner it is typically used in the patent context. Use of broader terms such as “comprises”, “includes”, and “having” should be understood to provide support for narrower terms such as “consisting of”, “consisting essentially of”, and “comprised substantially of”.

The phrases “in one embodiment,” “according to one embodiment,” and the like generally mean that the particular feature, structure, or characteristic following the phrase may be included in at least one embodiment of the present disclosure, and may be included in more than one embodiment of the present disclosure (importantly, such phrases do not necessarily refer to the same embodiment).

The word “example” or “exemplary” is used herein to mean “serving as an example, instance, or illustration.” Any implementation described herein as “exemplary” is not necessarily to be construed as preferred or advantageous over other implementations.

If the specification states a component or feature “may,” “can,” “could,” “should,” “would,” “preferably,” “possibly,” “typically,” “optionally,” “for example,” “often,” or “might” (or other such language) be included or have a characteristic, that a specific component or feature is not required to be included or to have the characteristic. Such a component or feature may be optionally included in some embodiments, or it may be excluded.

An electronic real-time health monitoring system (“RTHMS”) may be deployed to assist healthcare staff, such as nurses and hospital operations. An RTHMS may typically assist in providing “virtual care” by collecting certain patient vitals for continuous monitoring and coordinated care. An RTHMS may, in some examples, include a communication platform for remote and real-time monitoring of patients. For example, an RTHMS may be coupled to sensing hardware to capture vital parameters of patients in real-time and transmits data corresponding to the vital parameters to, for example, a cloud computing network, a remote computing platform, a computing network, one or more computers, and/or the like. The cloud computing network may perform analytics and monitoring of the data. Users of the RTHMS may retrieve, using client devices, the analytics and monitoring for rendering on, for example, a health analytics dashboard user interface on the client devices. The RTHMS may also generate alerts to the client devices in real-time based on any deviation in patients' vital signs against acceptable or normal ranges according to the analytics and monitoring.

Various example embodiments of the present disclosure provide various technical advancements and improvements to, in some examples, an RTHMS. In accordance with various examples of the present disclosure, integration of an internal organ temperature monitoring system with the RTHMS is disclosed. The disclosed internal organ temperature monitoring system may receive sensor data associated with an internal organ temperature sensor system, process the data to determine internal organ temperature characteristics at patient side, and transmit the internal organ temperature characteristics to the RTHMS. The disclosed internal organ temperature monitoring system may also be integrated with nurse call systems, such as to improve healthcare staff productivity, other hospital or remote systems, and/or other remote care providers.

According to some embodiments, an internal organ temperature monitoring system may be configured to monitor internal organ temperature. For example, the internal organ temperature may be monitored in real time during a critical surgical operation. For example, the internal organ temperature may also be monitored before the critical surgical operation and post the critical surgical operation. The internal organ temperature monitoring system may include a controller component capable of executing program code to monitor an internal organ temperature in real time, near-real time, and/or the like.

The disclosed internal organ temperature monitoring system may also be provided as a frontend device for an RTHMS to improve productivity and/or responsiveness, such as healthcare staff productivity, with high/low alerts and alarms via visual and acoustic indications on the sensor apparatus. The controller component of the internal organ temperature monitoring system may determine the internal organ temperature characteristics and transmit a data load package including the internal organ temperature characteristics for high/low alerts and alarms to an RTHMS. Data load packages may be transmitted to the RTHMS periodically or upon detection of the internal organ temperature characteristics.

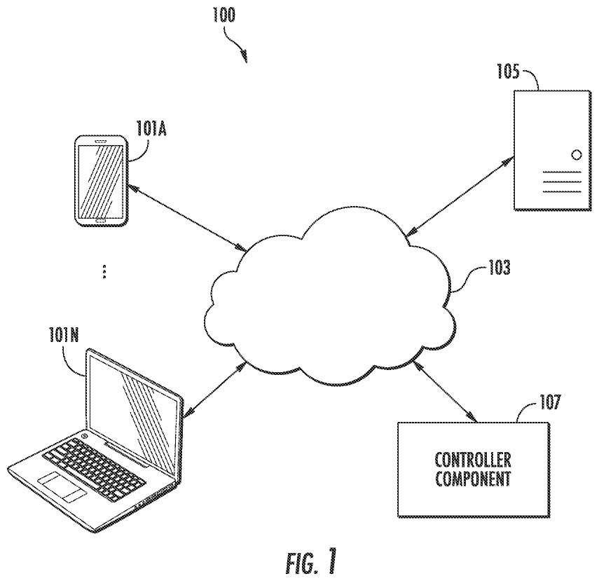

Referring now to FIG. 1, an example diagram illustrating an example analytics and monitoring system 100 in accordance with some example embodiments described herein is provided. As shown in FIG. 1, the example analytics and monitoring system 100 includes apparatuses, devices, and components such as, but not limited to, a controller component 107, one or more mobile devices 101A . . . 101N, a remote computing server 105 in a remote computing platform, and one or more networks 103.

In some embodiments, each of the components of the example analytics and monitoring system 100 may be in electronic communication with, for example, one another over the same or different wireless or wired networks 103 including, for example, a wired or wireless Personal Area Network (PAN), Local Area Network (LAN), Metropolitan Area Network (MAN), Wide Area Network (WAN), and/or the like. Additionally, while FIG. 1 illustrates certain system entities as separate, standalone entities, the various embodiments are not limited to this particular architecture.

For example, the controller component 107, one or more mobile devices 101A . . . 101N, and the remote computing server 105 in the remote computing platform may be in electronic communication with one another to exchange data and information. As described herein, the controller component 107 may receive data from one or more sensor systems. In some embodiments, the controller component 107 may transmit data from the one or more sensor systems to the one or more mobile devices 101A . . . 101N and/or the remote computing server 105 in the remote computing platform for analysis. For example, the one or more mobile devices 101A . . . 101N may be any terminal devices in the hospital workflow. Data from the one or more sensors may be distributed into individual monitoring channels corresponding to each of the sensors.

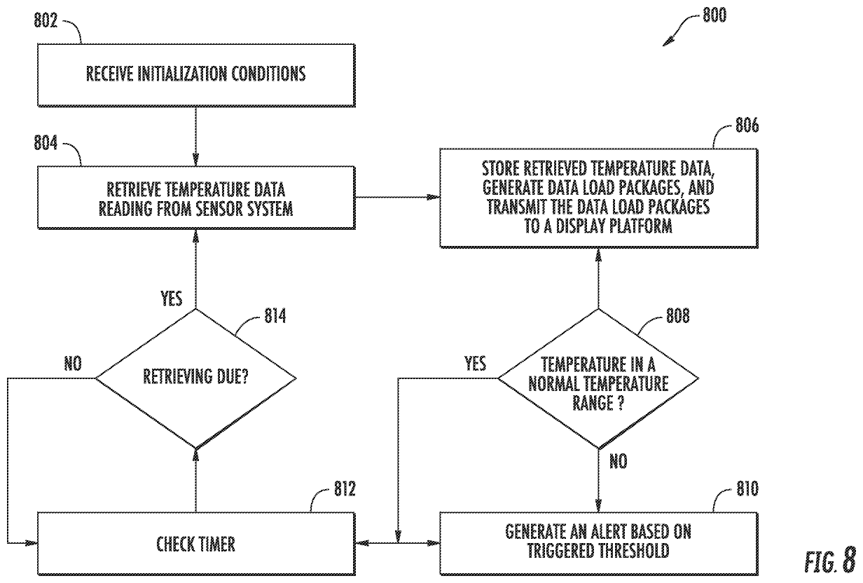

In some embodiments, the one or more mobile devices 101A . . . 101N and/or the remote computing server 105 in the remote computing platform may receive the data from the controller component 107 and may generate estimated characteristics data associated with the one or more sensor systems based at least in part on the data. For example, the controller component 107 may transmit data to the one or more mobile devices 101A . . . 101N and/or the remote computing server 105. Upon receiving the data, the one or more mobile devices 101A . . . 101N and/or the remote computing server 105 may process the estimated characteristics data for rendering on a graphical user interface (“GUI”). In some embodiments, the one or more mobile devices 101A . . . 101N and/or the remote computing server 105 may generate one or more data points on the GUI based on the data in accordance with various example methods described herein, including, but not limited to, those described in connection with FIG. 8.

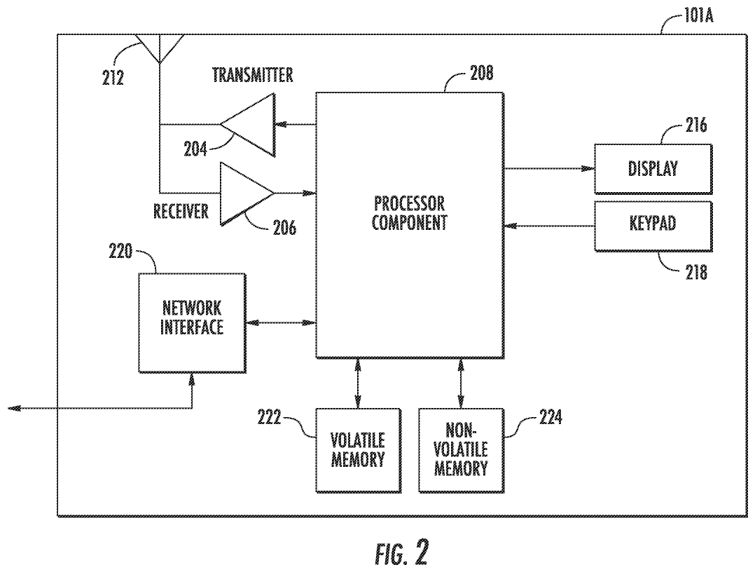

Referring now to FIG. 2, an example schematic representation of an example mobile device in accordance with some example embodiments described herein is provided. For example, FIG. 2 provides an illustrative schematic representative of one of the mobile devices 101A to 101N that can be used in conjunction with embodiments of the present disclosure. In some embodiments, as illustrated in FIG. 2, the mobile device 101A includes an antenna 212, a transmitter 204 (e.g., radio), a receiver 206 (e.g., radio), and a processor component 208 that provides signals to and receives signals from the transmitter 204 and receiver 206, respectively. The signals provided to and received from the transmitter 204 and the receiver 206, respectively, may include signaling information/data in accordance with an air interface standard of applicable wireless systems to communicate with various entities, such as a remote computing server 105, another mobile device 101A, an exemplary monitoring system and/or the like. In this regard, the mobile device 101A may be capable of operating with one or more air interface standards, communication protocols, modulation types, and access types. More particularly, the mobile device 101A may include a network interface 220, and may operate in accordance with any of a number of wireless communication standards and protocols. In a particular embodiment, the mobile device 101A may operate in accordance with multiple wireless communication standards and protocols, such as GPRS, UMTS, CDMA1900, 1×RTT, WCDMA, TD-SCDMA, LTE, E-UTRAN, EVDO, HSPA, HSDPA, Wi-Fi, WiMAX, UWB, IR protocols, Bluetooth protocols, USB protocols, and/or any other wireless protocol.

Via these communication standards and protocols, the mobile device 101A can communicate with various other entities using Unstructured Supplementary Service data (USSD), Short Message Service (SMS), Multimedia Messaging Service (MMS), Dual-Tone Multi-Frequency (DTMF) Signaling, Subscriber Identity Module Dialer (SIM dialer), and/or the like. The mobile device 101A can also download changes, add-ons, and updates, for instance, to its firmware, software (e.g., including executable instructions, applications, program modules), and operating system.

The mobile device 101A may also include a user interface comprising one or more user input/output interfaces (e.g., a display 216 and/or speaker/speaker driver coupled to a processor component 208 and a touch screen, keyboard, mouse, and/or microphone coupled to a processor component 208). For example, the user output interface may be configured to provide an application, browser, user interface, dashboard, webpage, and/or similar words used herein interchangeably executing on and/or accessible via the mobile device 101A to cause display or audible presentation of information/data and for user interaction therewith via one or more user input interfaces. The user output interface may be updated dynamically from communication with the remote computing server 105. The user input interface can include any of a number of devices allowing the mobile device 101A to receive data, such as a keypad 218 (hard or soft), a touch display, voice/speech or motion interfaces, scanners, readers, or other input device. In embodiments including a keypad 218, the keypad 218 can include (or cause display of) the conventional numeric (0-9) and related keys (#, *), and other keys used for operating the mobile device 101A and may include a full set of alphabetic keys or set of keys that may be activated to provide a full set of alphanumeric keys. In addition to providing input, the user input interface can be used, for example, to activate or deactivate certain functions, such as screen savers and/or sleep modes. Through such inputs the mobile device 101A can collect information/data, user interaction/input, and/or the like.

The mobile device 101A can also include volatile storage or memory 222 and/or non-volatile storage or memory 224, which can be embedded and/or may be removable. For example, the non-volatile memory may be ROM, PROM, EPROM, EEPROM, flash memory, MMCs, SD memory cards, Memory Sticks, CBRAM, PRAM, FeRAM, RRAM, SONOS, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. The volatile and non-volatile storage or memory can store databases, database instances, database management system entities, data, applications, programs, program modules, scripts, source code, object code, byte code, compiled code, interpreted code, machine code, executable instructions, and/or the like to implement the functions of the mobile device 101A-101N.

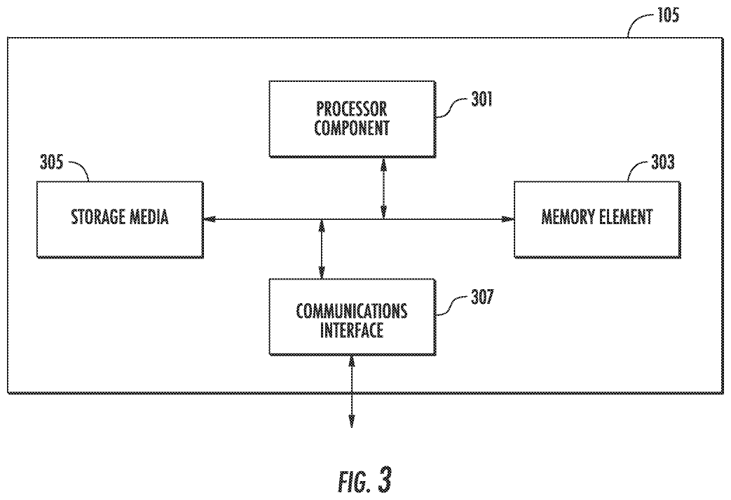

Referring now to FIG. 3, an example schematic representation of an example remote computing server 105 in an example remote computing platform in accordance with some example embodiments described herein. In some embodiments, the example remote computing platform may be a cloud computing platform, and the example remote computing server may be a cloud computing server.

As indicated, in some embodiments, the remote computing server 105 may include one or more network and/or communications interface 307 for communicating with various computing entities, such as by communicating data, content, information, and/or similar terms used herein that can be transmitted, received, operated on, processed, displayed, stored, and/or the like. For instance, the remote computing server 105 may communicate with controller component 107, one or more mobile devices 101A . . . 101N, and/or the like.

As shown in FIG. 3, in one embodiment, the remote computing server 105 may include or be in communication with one or more processor components (for example, processor component 301) (also referred to as processor components, processing circuitry, and/or similar terms used herein interchangeably) that communicate with other elements within the remote computing server 105 via a bus, for example, or network connection. As will be understood, the processor component 301 may be embodied in a number of different ways. For example, the processor component 301 may be embodied as one or more complex programmable logic devices (CPLDs), microprocessor components, multi-core processor components, co-processing entities, application-specific instruction-set processor components (ASIPs), and/or controllers. Further, the processor component 301 may be embodied as one or more other processing devices or circuitry. The term circuitry may refer to an entirely hardware embodiment or a combination of hardware and computer program products. Thus, the processor component 301 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processor component 301 may be configured for a particular use or configured to execute instructions stored in volatile or non-volatile media or otherwise accessible to the processor component 301. As such, whether configured by hardware or computer program products, or by a combination thereof, the processor component 301 may be capable of performing steps or operations according to embodiments of the present disclosure when configured accordingly.

In one embodiment, the remote computing server 105 may further include or be in communication with volatile media (also referred to as volatile storage, memory, memory storage, memory circuitry and/or similar terms used herein interchangeably). In one embodiment, the volatile storage or memory may also include one or more memory element 303 as described above, such as RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. As will be recognized, the volatile storage or memory element 303 may be used to store at least portions of the databases, database instances, database management system entities, data, applications, programs, program modules, scripts, source code, object code, byte code, compiled code, interpreted code, machine code, executable instructions, and/or the like being executed by, for example, the processor component 301 as shown in FIG. 3. Thus, the databases, database instances, database management system entities, data, applications, programs, program modules, scripts, source code, object code, byte code, compiled code, interpreted code, machine code, executable instructions, and/or the like may be used to control certain aspects of the operation of the remote computing server 105 with the assistance of the processor component 301 and operating system.

In one embodiment, the remote computing server 105 may further include or be in communication with non-volatile media (also referred to as non-volatile storage, memory, memory storage, memory circuitry and/or similar terms used herein interchangeably). In one embodiment, the non-volatile storage or memory may include one or more non-volatile storage or storage media 305 as described above, such as hard disks, ROM, PROM, EPROM, EEPROM, flash memory, MMCs, SD memory cards, Memory Sticks, CBRAM, PRAM, FeRAM, RRAM, SONOS, racetrack memory, and/or the like. As will be recognized, the non-volatile storage or storage media 305 may store databases, database instances, database management system entities, data, applications, programs, program modules, scripts, source code, object code, byte code, compiled code, interpreted code, machine code, executable instructions, and/or the like. The term database, database instance, database management system entity, and/or similar terms used herein interchangeably and in a general sense to refer to a structured or unstructured collection of information/data that is stored in a computer-readable storage medium.

Storage media 305 may also be embodied as a data storage device or devices, as a separate database server or servers, or as a combination of data storage devices and separate database servers. Further, in some embodiments, storage media 305 may be embodied as a distributed repository such that some of the stored information/data is stored centrally in a location within the system and other information/data is stored in one or more remote locations. Alternatively, in some embodiments, the distributed repository may be distributed over a plurality of remote storage locations only. An example of the embodiments contemplated herein would include a cloud data storage system maintained by a third-party provider and where some or all of the information/data required for the operation of the recovery prediction system may be stored.

As indicated, in one embodiment, the remote computing server 105 may also include one or more network and/or communications interface 307 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the remote computing server 105 may be configured to communicate via wireless external communication networks using any of a variety of protocols, such as general packet radio service (GPRS), Universal Mobile Telecommunications System (UMTS), Code Division Multiple Access 1900 (CDMA1900), CDMA1900 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), Institute of Electrical and Electronics Engineers (IEEE) 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol. The remote computing server 105 may use such protocols and standards to communicate using Border Gateway Protocol (BGP), Dynamic Host Configuration Protocol (DHCP), Domain Name System (DNS), File Transfer Protocol (FTP), Hypertext Transfer Protocol (HTTP), HTTP over TLS/SSL/Secure, Internet Message Access Protocol (IMAP), Network Time Protocol (NTP), Simple Mail Transfer Protocol (SMTP), Telnet, Transport Layer Security (TLS), Secure Sockets Layer (SSL), Internet Protocol (IP), Transmission Control Protocol (TCP), User Datagram Protocol (UDP), Datagram Congestion Control Protocol (DCCP), Stream Control Transmission Protocol (SCTP), HyperText Markup Language (HTML), and/or the like.

As will be appreciated, one or more of the remote computing server's components may be located remotely from components of other remote computing servers, such as in a distributed system. Furthermore, one or more of the components may be aggregated and additional components performing functions described herein may be included in the remote computing server 105. Thus, the remote computing server 105 can be adapted to accommodate a variety of needs and circumstances.

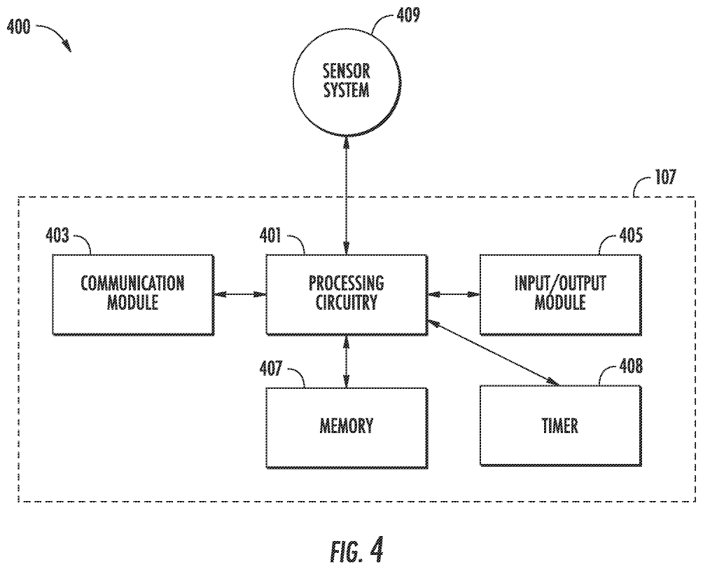

Referring now to FIG. 4, a schematic diagram depicting an exemplary internal organ temperature monitoring system in accordance with various embodiments of the present disclosure. As shown, an internal organ temperature monitoring system 400 may include the controller component 107 and a sensor system 409. The controller component 107 includes processing circuitry 401, a communication module 403, input/output module 405, a memory 407, a timer 408, and/or other components configured to perform various operations, procedures, functions or the like described herein.

As shown, the controller component 107 (such as the processing circuitry 401, communication module 403, input/output module 405 and memory 407) is electrically coupled to and/or in electronic communication with the sensor system 409. As depicted, the sensor system 409 may exchange (e.g., transmit and receive) data with the processing circuitry 401 of the controller component 107. For example, the sensor system 409 may generate sensor data and transmit the sensor data to the processing circuitry 401.

The processing circuitry 401 may be implemented as, for example, various devices comprising one or a plurality of microprocessors with accompanying digital signal processors; one or a plurality of processors without accompanying digital signal processors; one or a plurality of coprocessors; one or a plurality of multi-core processors; one or a plurality of controllers; processing circuits; one or a plurality of computers; and various other processing elements (including integrated circuits, such as ASICs or FPGAs, or a certain combination thereof). In some embodiments, the processing circuitry 401 may include one or more processors. In one exemplary embodiment, the processing circuitry 401 is configured to execute instructions stored in the memory 407 or otherwise accessible by the processing circuitry 401. When executed by the processing circuitry 401, these instructions may enable the controller component 107 to execute one or a plurality of the functions as described herein. No matter whether it is configured by hardware, firmware/software methods, or a combination thereof, the processing circuitry 401 may include entities capable of executing operations according to the embodiments of the present invention when correspondingly configured. Therefore, for example, when the processing circuitry 401 is implemented as an ASIC, an FPGA, or the like, the processing circuitry 401 may include specially configured hardware for implementing one or a plurality of operations described herein. Alternatively, as another example, when the processing circuitry 401 is implemented as an actuator of instructions (such as those that may be stored in the memory 407), the instructions may specifically configure the processing circuitry 401 to execute one or a plurality of algorithms and operations described herein.

The memory 407 may include, for example, a volatile memory, a non-volatile memory, or a certain combination thereof. Although illustrated as a single memory in FIG. 4, the memory 407 may include a plurality of memory components. In various embodiments, the memory 407 may include, for example, a hard disk drive, a random-access memory, a cache memory, a flash memory, a Compact Disc Read-Only Memory (CD-ROM), a Digital Versatile Disk Read-Only Memory (DVD-ROM), an optical disk, a circuit configured to store information, or a certain combination thereof. The memory 407 may be configured to store information, data, application programs, instructions, and etc., so that the controller component 107 can execute various functions according to the embodiments of the present disclosure. For example, in at least some embodiments, the memory 407 is configured to cache input data for processing by the processing circuitry 401. Additionally, or alternatively, in at least some embodiments, the memory 407 is configured to store program instructions for execution by the processing circuitry 401. The memory 407 may store information in the form of static and/or dynamic information. When the functions are executed, the stored information may be stored and/or used by the controller component 107.

In some embodiments, the timer 408 may be a clock, which is used to measure time intervals and determine time elapsed. For example, a timer may be used to in multi-threaded operating systems to determine how long a task is active before swapping to a new task. For example, a timer may also be used to determine a retrieving rate of a signal.

The communication module 403 may be implemented as any apparatus included in a circuit, hardware, a computer program product, or a combination thereof, which is configured to receive and/or transmit data from/to another component or apparatus. The computer program product includes computer-readable program instructions stored on a computer-readable medium (for example, the memory 407) and executed by a controller component 107 (for example, the processing circuitry 401). In some embodiments, the communication module 403 (as with other components discussed herein) may be at least partially implemented as the processing circuitry 401 or otherwise controlled by the processing circuitry 401. In this regard, the communication module 403 may communicate with the processing circuitry 401, for example, through a bus. The communication module 403 may include, for example, antennas, transmitters, receivers, transceivers, network interface cards and/or supporting hardware and/or firmware/software, and is used for establishing communication with another apparatus. The communication module 403 may be configured to receive and/or transmit any data that may be stored by the memory 407 by using any protocol that can be used for communication between apparatuses. The communication module 403 may additionally or alternatively communicate with the memory 407, the input/output module 405 and/or any other component of the controller component 107, for example, through a bus.

In some embodiments, the controller component 107 may include an input/output module 405. The input/output module 405 may communicate with the processing circuitry 401 to receive instructions input by the user and/or to provide audible, visual, mechanical, or other outputs to the user. Therefore, the input/output module 405 may include supporting devices, such as a keyboard, a mouse, a display, a touch screen display, and/or other input/output mechanisms. Alternatively, at least some aspects of the input/output module 405 may be implemented on a device used by the user to communicate with the controller component 107. The input/output module 405 may communicate with the memory 407, the communication module 403 and/or any other component, for example, through a bus. One or a plurality of input/output modules and/or other components may be included in the controller component 107.

In some embodiments, the controller component 107 may include a timer 408. For example, the timer may be a clock, which is used to measure time intervals and determine time elapsed. For example, a timer may be used to in multi-threaded operating systems to determine how long a task is active before swapping to a new task. For example, a timer may also be used to determine a retrieving rate of a signal.

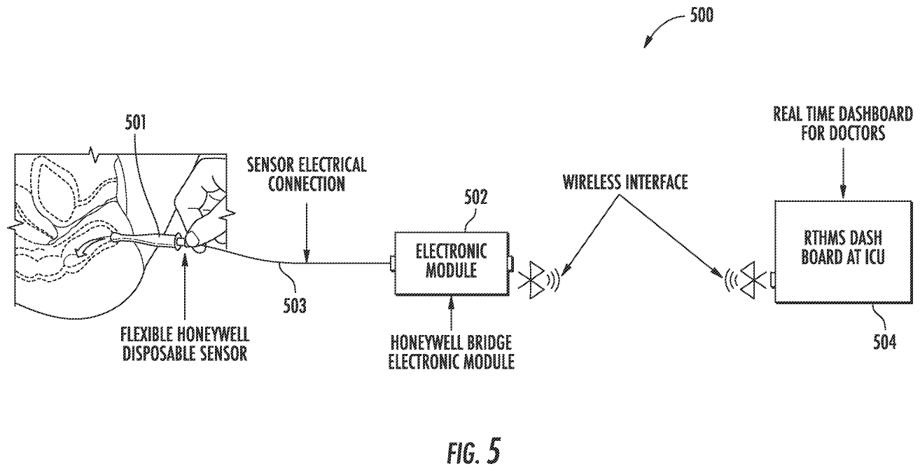

Referring now to FIG. 5, an internal organ temperature monitoring system is provided, which may be used in accordance with various embodiments of the present disclosure. In some embodiments, the internal organ temperature monitoring system 500 may include a sensor system 501, a controller component 502, and an electrical cable 503 electrically connecting the sensor system 501 and the controller component 502. The sensor system 501 is a temperature measuring probe, which may be inserted into an internal organ to measure the temperature of the internal organ. For example, the internal organ may be a rectum, an oral cavity, or an artery.

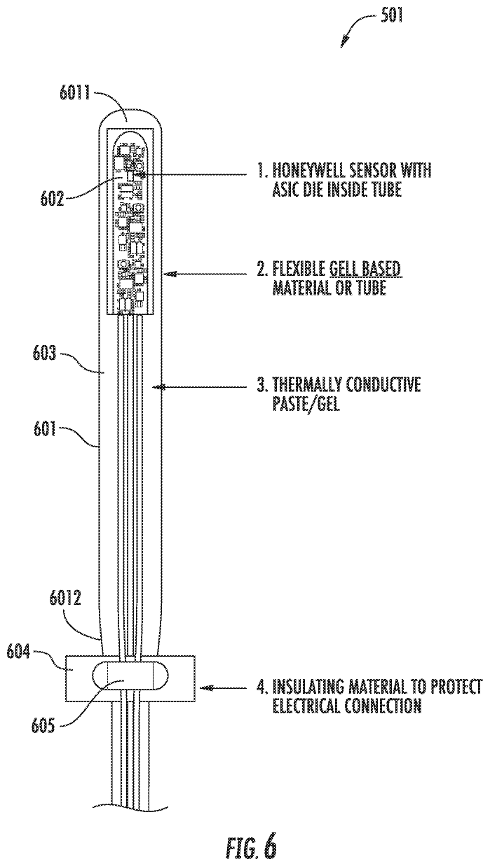

In some example embodiments, the sensor system 501 may be a flexible biocompatible probe. In particular, the sensor system 501 may be made of a material compatible with living tissues, such that no toxic substance is produced when the internal organ is exposed to the sensor system. In some examples embodiments, the sensor system 501 is replaceable. For example, the sensor system may be disposed after each use or a threshold number times of usages, and replaced with a new sensor system. In particular, the sensor system 501 may have a connector at one end and the electrical cable 503 may include a plug that is configured to be inserted into the connector. In some embodiments, the sensor system 501 may be replaced by plugging or otherwise attaching the electrical cable 503 into a connector of a new or replacement sensor system 501. Additional details regarding the sensor system 501 are further described with respect to FIG. 6.

The controller component 502, in some examples may be an electrical module. In particular, the controller component 502 may take the form of a Honeywell Bridge Electronic Module. For example, the Honeywell Bridge Electronic Module may be a bridge interface electronic controller module or an interface electronic controller module, which may be configured to initialize, configure, or calibrate the sensor system 501. The Honeywell Bridge Electronic Module may be further configured to perform data computations, write data into the sensor system 501, or read from sensor system 501. The Honeywell Bridge Electronic Module may also communicate as well as manage the hospital workflows. In some embodiments, the controller component 502 may be configured to retrieve the temperature data from the sensor system 501.

In some embodiments, the controller component 502 may calibrate the sensor of the sensor system 501 in an instance in which the sensor system 501 is connected with the controller component 502 for the first time. For example, algebraic difference may exist between a measured temperature of the sensor and the actual temperature. For example, the sensor system 501 may be inserted into a medium or organ with a known reference temperature. In particular, the medium may be an iced water bath with a temperature at 0° C., and the measured temperature from the sensor system 501 may be calibrated to be 0° C. In some embodiments, a medium with a different reference temperature, for example, a boiled water bath with a temperature at 100° C., may be used to calibrate the sensor system again. Calibration may eliminate the algebraic difference and make sure that the actual value of the temperature is sensed by the sensor system 501 and passed to the controller component 502.

In some examples, the controller component 502 may include an intermediate communication aggregator (e.g., a gateway) to a central network or a remote server. The controller component 502 may interface with sensor system 501 to retrieve the temperature data and process the temperature data. The controller component 502 may include a storage device to store the temperature data.

Based on at least the temperature data, the controller component 502 may create data load packages including the retrieved temperature data. The data load packages may be a standardized type of data that may be transmitted between and commonly supported by the controller component 502 and other display platforms, such as the display platforms described herein (e.g., RTHMS). The data load packages may further include information, such as device tag number/identified, alert/alarm flags with time stamps and optionally configuration packet to set various control logic values. For example, an example data load package may include the retrieved temperature data, an identifier of the sensor system, a device tag number of the controller, and a time stamp of retrieving the temperature data.

The data load packages may be transmitted by controller component 502 to the display platform 504, the remote computing server 105, or the mobile devices 101 over a wired or wireless network. The controller component 502 may also include physical buttons to initiate and stop monitoring of the internal temperature. The controller component 502 may further include visual/acoustic indicators to show respective alerts or alarms with respect to monitoring characteristics (e.g., a measured temperature value is out of a normal temperature range).

The internal organ temperature monitoring system 500 may further include a display platform 504, in some examples, which may communicate with the controller component 502 by transmitting sensor data from the controller component 502 to the display platform 504.

In some examples, the display platform 504 may be a cloud/remote monitoring system, remote computing platform, a computing network, one or more computers, and/or the like. In particular, the display platform 504 may be a real-time health monitoring system (RTHMS). The RTHMS may communicate with the controller component 502 through wireless or wired communication means. The sensing mechanism through the sensor system 501 is to connect with the controller component 502 to process the signal and send the processed data through wired/wireless means into cloud/remote display platforms, such as an RTHMS, hospital workflows, or mobile devices).

According to various example embodiments of the present disclosure, the display platform 504 may be an RTHMS, and the RTHMS may include software framework hosted, e.g., on remote computing server 105, configured to receive data load packages as described above to process value added services in form of timely communications to healthcare staff, caregivers, hospital staff, nurses, or the like about alarms over mobile device dashboards, or provide monitoring information along with hospital enterprise level data visualization or analytics. The RTHMS may further include an overall workflow controller that may provide nurse or other caregiver call functions, mobile dashboard, and enterprise rule engines to qualify alerts/alarms. In one embodiment, the RTHMS may manage a nurse call system in response to the temperature monitoring related workflows.

FIG. 6 illustrates an exemplary replaceable internal organ temperature sensor system in accordance with various embodiments of the present disclosure. As shown in FIG. 6, a sensor system 501 may be interchangeably coupled to a controller component via an electrical cable (e.g., the controller component 502 via the electrical cable 503 as shown in FIG. 5). According to certain example embodiments of the present disclosure, the sensor system 501 may include a tube 601, a sensor 602 embedded inside the tube 601 and located at a first end 6011 of the tube 601, and a thermal conductive material 603 that is configured to fill or otherwise occupy the space between the sensor 602 and the tube 601. The sensor system 501 may further include an electrical connector 605 located at the second end 6012 of the tube 601, and an insulating seal 604 sealing up the electrical connector 605. The electrical connector 605 may couple the sensor 602 to the controller component (e. g. the controller component 502 as shown in FIG. 5).

In some embodiments, the tube 601 may be made of a flexible gel-based material. In some embodiments, the tube 601 may take the form of a catheter, which is a thin and flexible tube. The catheter may be approved by Food and Drug Administration (FDA).

In some embodiments, the sensor 602 may be a temperature sensor, which may produce an electrical signal according to the temperature around the sensor. For example, the sensor 602 may be an active type sensor and the active type temperature sensor may be powered by the controller component 502. The sensor 602 may convert the measured temperature into a digital signal and thus communicate with the controller component 502. When an active temperature sensor is exposed to an environment, an electronic signal may be produced according to the temperature of the environment. When the temperature of the environment changes, the electronic signal may change accordingly, and the electronic signal is directly corresponding to the temperature of the environment. In some embodiments, the sensor 602 may take the form of an Application specific integrated circuits (ASIC) die. The sensor 602 is powered by the controller component 502.

Alternatively, the sensor 602 may be a passive type temperature sensor, such as a thermocouple or a thermistor. In some examples, a passive type temperature sensor does not need a power to function. For example, a thermistor may include a material with a resistance correlated to a temperature of the environment. The temperature may be measured indirectly by measuring the resistance of the thermistor.

In some embodiments, the sensor 602 may have a sensitivity in a range of 0.1-0.6° C., more preferably in a range of 0.1-0.3° C., and most preferably of 0.2° C. In some embodiments, a better accuracy of temperature measurements may be achieved by a customized temperature sensor.

In some embodiments, the thermal conductive material 603 may be configured to fill or otherwise occupy the space between the sensor 602 and the tube 601 to provide thermal contact between the sensor 602 and the tube 601. Thermal conductive material 603 is made of a material that transports the heat across a temperature gradient due to random molecular/atomic motion. In some embodiments, the thermal conductive material 603 may be a thermal conductive gel. The thermal conductive material 603 may be in liquid formable and curable formulations, such that gaps or voids in the space between the sensor 602 and the tube 601 may be minimized while maximizing thermal performance between the sensor 602 and the tube 601. For example, the thermal conductive material 603 may be a silicone gel. As a result, the sensor 602 may stabilize to a temperature that is same as the temperature of the internal organ. For example, the sensor 602 may be able to stabilize to the temperature of the internal organ within 1-2 minutes when the sensor system is inserted into the internal organ for the first time.

In some embodiments, the insulating seal 604 may protect the electrical connector 605 of the sensor system 501. In some embodiments, the insulating seal 604 may insulate the tube 601 from the ambient environment outside the internal organ, such that an accuracy of the temperature measurement is not influenced by the ambient environment.

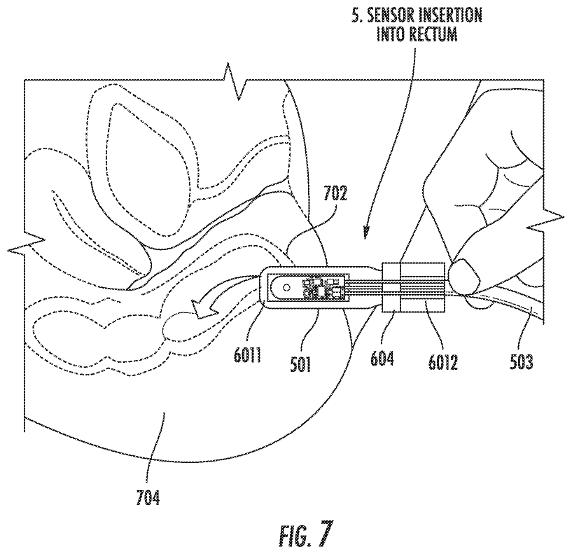

FIG. 7 illustrates an exemplary diagram 700 illustrating an internal organ temperature sensor system inserted into an internal organ, in accordance with various embodiments of the present disclosure. As shown in FIG. 7, an exemplary sensor system 501 may be inserted into an internal organ 702 of a body 704. For example, the sensor system 501 may include a first end 6011 and a second end 6012, where the first end 6011 is inserted into the internal organ 702 and the second end 6012 is electrically connected to a controller component through an electrical cable 503. As an example, the first end 6011 of the system 501 is inserted into the internal organ until an insulating seal 604 is able to insulate at least the first end 6011 of the sensor system 501 from the ambient environment. For example, when the sensor system 501 is inserted into the internal organ for the first time, the sensor system 501 may only take 1-2 minutes to stabilize to the temperature of the internal organ. In some embodiments, the internal organ may be a rectum. In some embodiments, the internal organ may be an oral cavity or an artery.

Referring now to FIG. 8, an exemplary flow diagram illustrating an exemplary method of monitoring internal organ temperature by a controller component in accordance with some exemplary embodiments of the present disclosure are provided. It is noted that each block of a flowchart, and combinations of blocks in the flowchart, may be implemented by various means such as hardware, firmware, circuitry and/or other devices associated with execution of software including one or more computer program instructions. For example, one or more of the steps/operations described in FIG. 8 may be embodied by computer program instructions, which may be stored by a non-transitory memory of an apparatus employing an embodiment of the present disclosure and executed by a processor component in an apparatus (such as, but not limited to, a controller component, a programmable processor, a mobile device, a remote computing server, and/or the like). For example, these computer program instructions may direct the processor component to function in a particular manner, such that the instructions stored in the computer-readable storage memory produce an article of manufacture, the execution of which implements the function specified in the flowchart block(s).

As described above and as will be appreciated based on this disclosure, embodiments of the present disclosure may include various means including entirely of hardware or any combination of software and hardware. Furthermore, embodiments may take the form of a computer program product on at least one non-transitory computer-readable storage medium having computer-readable program instructions (e.g., computer software) embodied in the storage medium. Similarly, embodiments may take the form of a computer program code stored on at least one non-transitory computer-readable storage medium. Any suitable computer-readable storage medium may be utilized including non-transitory hard disks, CD-ROMs, flash memory, optical storage devices, or magnetic storage devices.

Referring now to FIG. 8, an exemplary method 800 for monitoring an internal organ temperature in accordance with some exemplary embodiments described herein is illustrated. The exemplary method 800 may be executed by a computing device associated with a controller component (for example, the controller component 502 as illustrated and described above in connection with at least FIG. 5) including processing circuitry and memory (for example, the processing circuitry 401 and the memory 407 as illustrated and described above in connection with at least FIG. 4).

At step 802, the controller component may receive data representative of initialization conditions. As an example, the controller component may include an interface (e.g., input/output module 405) for entering the initialization conditions. The initialization conditions may include a normal temperature range of the internal organ. For example, the normal temperature for a human body may be in a range from 36.1° C. to 37.2° C. In particular, the normal temperature range may vary according to different medical conditions, such as diseases, medicines used, or surgical operations performed. The initialization conditions may further include a retrieving rate. For example, the controller component 502 may read the temperature from the sensor system once for every five seconds. In some embodiments, the controller component 502 may read the temperature from the sensor system continuously.

In some embodiments, at step 804, the controller component is configured to retrieve temperature data reading from the sensor system coupled to the controller component. For example, the sensor system may include a temperature sensor configured to measure the temperature data of an internal organ.

In some embodiments, at step 806, the controller component stores the retrieved temperature data. In particular, the controller component may store at least values for an initial temperature data and a temperature data of a current environment. For example, the controller component may use a numerical display on the controller component to display the retrieved temperature data. The controller component may further communicate and transmit the retrieved temperature data to the display platform (e.g., an RTHMS). In some embodiments, at step 806, the controller component may further generate data load packages, which may include the retrieved temperature data, an identifier of the sensor system, a device tag number of the controller, and a time stamp of retrieving the temperature data. The controller component may further transmit the data load packages to the display platform 504. In some embodiments, the controller component may generate and transmit the data load packages in real time, near-real time, and/or the like. In some examples, the display platform 504 may be a cloud/remote monitoring system, remote computing platform, a computing network, one or more computers, and/or the like. In particular, the display platform 504 may be a real-time health monitoring system (RTHMS). The RTHMS may communicate with the controller component 502 through wireless or wired communication means. The sensing mechanism through the sensor system 501 is to connect with the controller component 502 to process the signal and send the processed data through wired/wireless means into cloud/remote display platforms, such as an RTHMS, hospital workflows, or mobile devices).

In some embodiments, at step 808, the controller component determines whether the retrieved temperature data falls within the normal temperature range. Alert thresholds and alarm thresholds may be configured, for example, to suit response protocols. The alert thresholds and the alarm thresholds may be configured in terms of the normal temperature range. For example, an alert may be configured at the boundary of the normal temperature range. In particular, the normal temperature range may be within 5% of an average of the normal internal organ temperature. If the retrieved temperature data falls in the normal temperature range, the example method may proceed to step 812.

In some embodiments, if the retrieved temperature data does not fall in the normal temperature range, the exemplary method may proceed to step 810, where the controller component generates an alert or an alarm based on the retrieved temperature data. For example, the alerts or alarms may be high/low alerts or alarms via visual and acoustic indications on the controller component. For example, a visual indication may include a text indicator, a color indicator, an icon indicator, or a combination of a color indicator and an icon indicator over a remote display system or an LED based on a remote device. The visual indication may be an indicator along with any specified acoustic indications acceptable in hospitals or end user related environment. In some embodiments, the controller component 502 process the signal and send the processed data through wired/wireless means into cloud/remote monitoring systems, such as an RTHMS, hospital workflows, or mobile devices).

In some embodiments, at step 812, the controller component may check a timer of the controller component. For example, the timer may be a clock, which is used to measure time intervals and determine time elapsed. In particular, the timer may be used to determine retrieving rate for the temperature data readings.

In some embodiments, at step 814, the controller component determines whether temperature data reading from the sensor system is due. For example, the controller component may determine, according to the retrieving rate, whether a temperature data reading is due. According to some embodiments, retrieving rate of the sensor system may be predetermined or programmable. For example, when the retrieving rate is initialized at step 802 as one reading for every five seconds, the controller component may determine at specific intervals, such as after every five seconds, to proceed to step 804 to retrieve the temperature data from the sensor system. If retrieving is due, the exemplary method may proceed to step 804, where the controller component retrieves temperature data reading from the sensor. Otherwise, the exemplary method proceeds to step 812, where the controller component checks the timer.

As described above and as will be appreciated based on this disclosure, embodiments of the present disclosure may include various means including entirely of hardware or any combination of software and hardware. Furthermore, embodiments may take the form of a computer program product on at least one non-transitory computer-readable storage medium having computer-readable program instructions (e.g., computer software) embodied in the storage medium. Similarly, embodiments may take the form of a computer program code stored on at least one non-transitory computer-readable storage medium. Any suitable computer-readable storage medium may be utilized including non-transitory hard disks, CD-ROMs, flash memory, optical storage devices, or magnetic storage devices.

It is to be understood that the disclosure is not to be limited to the specific embodiments disclosed, and that modifications and other embodiments are intended to be included within the scope of the appended claims. Although specific terms are employed herein, they are used in a generic and descriptive sense only and not for purposes of limitation, unless described otherwise.

What is claimed is:

1. An internal organ temperature monitoring system, comprising:
a sensor system comprising:
a tube having a first end and a second end;
a temperature sensor in the tube and located at the first end of the tube;
a thermal conductive material occupying space between the temperature sensor and the tube; and
an electrical connector located at the second end of the tube, coupling the sensor to a controller component; and
the controller component configured to retrieve temperature data from the temperature sensor.

2. The internal organ temperature monitoring system of claim 1, wherein the controller component is further configured to transmit data load packages to a remote computing server, a real-time health monitoring system (RTHMS), hospital workflows, or mobile devices, the data load packages including the retrieved temperature data.

3. The internal organ temperature monitoring system of claim 1, wherein the controller component further includes visual and acoustic indicators that show respective alerts or alarms with respect to the retrieved temperature data.

4. The internal organ temperature monitoring system of claim 3, wherein the controller component is further configured to store the retrieved temperature data and show the respective alerts or alarms in an instance in which the retrieved temperature data falls out of a normal temperature range.

5. The internal organ temperature monitoring system of claim 1, wherein the temperature sensor is an active type sensor with an application specific integrated circuits (ASIC) die, and the active type sensor is powered by the controller component.

6. The internal organ temperature monitoring system of claim 1, wherein the temperature sensor is a passive type sensor, and the passive type sensor is a thermocouple or a thermistor.

7. The internal organ temperature monitoring system of claim 1, wherein the tube takes a form of a catheter made of a flexible gel-based material.

8. The internal organ temperature monitoring system of claim 1, wherein the sensor system further comprises an insulating seal to insulate the electrical connector and the tube from an ambient environment outside an internal organ, wherein the insulating seal is located at the second end of the tube.

9. The internal organ temperature monitoring system of claim 1, wherein the sensor system is configured to be inserted into an internal organ to measure a temperature of the internal organ.

10. The internal organ temperature monitoring system of claim 1, wherein the sensor system is replaceable.

11. A method for monitoring an internal organ temperature by a controller component, comprising:
retrieving temperature data reading from a sensor system, wherein the sensor system comprising a tube having a first end and a second end; a temperature sensor in the tube and located at the first end of the tube; a thermal conductive material occupying space between the temperature sensor and the tube; and an electrical connector located at the second end of the tube, coupling the sensor to the controller component;
determining whether the retrieved temperature data is in a normal temperature range;
generating an alert in an instance in which the retrieved temperature data is not in the normal temperature range.

12. The method for monitoring the internal organ temperature of claim 11, further comprising transmitting data load packages to a remote computing server, a real-time health monitoring system (RTHMS), hospital workflows, or mobile devices, wherein the data load packages include the retrieved temperature data.

13. The method for monitoring the internal organ temperature of claim 11, further comprising:
receiving initialization conditions, by the controller component, before retrieving the temperature data reading from the sensor system, wherein the initialization conditions include a retrieving rate and the normal temperature range.

14. The method for monitoring the internal organ temperature of claim 13, wherein the controller component is further configured to store the retrieved temperature data.

15. The method for monitoring the internal organ temperature of claim 11, wherein the temperature sensor is an active type sensor with an application specific integrated circuits (ASIC) die, and the active type sensor is powered by the controller component.

16. The method for monitoring the internal organ temperature of claim 11, wherein the temperature sensor is a passive type sensor, and the passive type sensor is a thermocouple or a thermistor.

17. The method for monitoring the internal organ temperature of claim 11, wherein the tube takes a form of a catheter made of a flexible gel-based material.

18. The method for monitoring the internal organ temperature of claim 11, wherein the sensor system further comprises an insulating seal to insulate the electrical connector and the tube from an ambient environment outside an internal organ, wherein the insulating seal is located at the second end of the tube.

19. The method for monitoring the internal organ temperature of claim 11, further comprising inserting the sensor system into an internal organ before retrieving the temperature data reading from the sensor system.

20. The method for monitoring the internal organ temperature of claim 11, wherein the sensor system is replaceable.
